(19) United States
(12) Patent Application Publication
(10) Pub. No.: US 2008/0206096 A1
(43) Pub. Date: Aug. 28, 2008

(54) Medical packaging substrate for ozone sterilization

(76) Inventor: Ganesh C. Deka, Duluth, GA (US)

Correspondence Address: DORITY & MANNING, P.A., POST OFFICE BOX 1449, GREENVILLE, SC, 29602-1449, US

(21) Appl. No.: 11/711,495

(22) Filed: Feb. 27, 2007

(51) Int. Cl. A61L 2/16; B05D 3/10; B32B 5/18

(52) U.S. Cl. 422/28; 427/299; 428/221

(57) ABSTRACT

A medical packaging substrate that is able to withstand ozone sterilization without generating a significant amount of odor and without undergoing a substantial degradation in strength is provided. This is accomplished by selectively controlling the components of the medical packaging substrate to optimize ozone compatibility. For example, the medical packaging substrate may be formed from a cellulosic fibrous material having a pH of about 7.0 or more. The present inventor has surprisingly discovered that such high pH values may allow the substrate to be effectively sterilized with ozone without generating substantial amounts of odor. In addition, a wet-strength agent and/or binder composition may also be selected that optimize the strength properties of the substrate without resulting in substantial odor.

### BACKGROUND OF THE INVENTION

Many products, especially devices and supplies used in surgical and other medical applications, must be sterilized prior to use. Examples of such products in the medical context include, but are not limited, to surgical devices, implants, tubing, valves, gauzing, and syringes. One sterilization procedure involves using sterilizing gases that will penetrate pores in a medical packaging substrate. The substrate may serve to protect contents during sterilization and to preserve their sterility upon subsequent storage until the packages are opened for use of the product. Medical packaging substrates may be used to package new medical items, as well as to wrap items such as surgical gowns, drapes, instruments, etc. for re-sterilization prior to reuse. Such sterilization wraps and their use are further described, for example, in U.S. Pat. Nos. 6,537,932, which is incorporated herein in its entirety by reference thereto for all purposes.

Steam and ethylene oxide are examples of suitable sterilizing gases. The gas flows through the pores in the substrate. Another method of sterilization uses ozone gas as a sterilizing agent. In ozone sterilization, oxygen is typically subjected to an electrical field to generate ozone (O₃), and thereafter humidified to improve sterilization efficacy. Ozone sterilization methods may be performed at ambient temperatures, which reduces the need for heating devices and permits use of less ozone. Using lower temperatures is also an advantage in that ozone is temperature sensitive and decomposes rapidly at higher temperatures. In addition, most ozone sterilization processes do not result in toxic waste, nor do they require the handling of dangerous gas cylinders. One particular ozone sterilization apparatus is TSO₃-125L, which is available from TSO₃, Inc. of Quebec City, Canada. Despite the benefits provided, however, the nature of conventional medical packaging substrates often limits the use of ozone sterilization. That is, the medical packaging substrates are typically made from Kraft and latex-impregnated cellulosic webs. Unfortunately, the cellulosic fibers of the substrate sometimes react with the highly oxidative ozone gas to produce odorous compounds.

As such, a need currently exists for an improved medical packaging substrate that is specifically tailored for ozone sterilization techniques.

### SUMMARY OF THE INVENTION

In accordance with one embodiment of the present invention, a medical packaging substrate is disclosed that comprises a fibrous web. The web is formed from a cellulosic fibrous material treated with a pH modifier so that the pH of the material is about 7.0 or more. The fibrous web is also impregnated with a binder composition. The impregnated fibrous web has a Gurley porosity of from about 10 to about 120 seconds per 100 cubic centimeters.

In accordance with another embodiment of the present invention, a method for forming a medical packaging substrate is disclosed. The method comprises forming a suspension into a fibrous web, the suspension comprising a cellulosic fibrous material. The fibrous web is impregnated with a binder composition. The suspension, the fibrous web prior to impregnation with the binder composition, or both are treated with a pH modifier so that the pH of the cellulosic fibrous material is about 7.0 or more.

In accordance with still another embodiment of the present invention, a method for sterilizing an item is disclosed. The method comprises enclosing the item within a medical package, wherein the medical package is formed from a substrate that comprises a cellulosic fibrous material that is impregnated with a binder composition. The substrate exhibits an initial machine direction and cross machine direction tensile strength. The method further comprises treating the substrate with ozone, wherein the ozone-treated substrate exhibits a machine direction tensile strength that is no more than about 40% less than the initial machine direction tensile strength.

Other features and aspects of the present invention are discussed in greater detail below.

### BRIEF DESCRIPTION OF THE DRAWINGS

A full and enabling disclosure of the present invention, including the best mode thereof to one of ordinary skill in the art, is set forth more particularly in the remainder of the specification, including reference to the accompanying figure in which:

FIG. 1 is a schematic illustration of one embodiment of an ozone sterilization apparatus that may be used in the present invention.

Repeat use of reference characters in the present specification and/or drawing is intended to represent same or analogous features or elements of the present invention.

### DETAILED DESCRIPTION OF REPRESENTATIVE EMBODIMENTS

It is to be understood by one of ordinary skill in the art that the present discussion is a description of exemplary embodiments only, and is not intended as limiting the broader aspects of the present invention, which broader aspects are embodied in the exemplary construction.

Generally speaking, the present invention is directed to a medical packaging substrate that is able to withstand ozone sterilization without generating a significant amount of odor and without undergoing a substantial degradation in strength. This is accomplished by selectively controlling the components of the medical packaging substrate to optimize ozone compatibility. For example, the medical packaging substrate may be formed from a cellulosic fibrous material having a pH of about 7.0 or more. The present inventor has surprisingly discovered that such high pH values may allow the substrate to be effectively sterilized with ozone without generating substantial amounts of odor. In addition, a wet-strength agent and/or binder composition may also be selected that optimize the strength properties of the substrate without resulting in substantial odor. Various embodiments of the present invention will now be described in more detail.

The medical packaging substrate of the present invention is formed from a fibrous web that contains a cellulosic fibrous material. As used herein, the term “cellulosic fibrous material” generally refers to a material that contains wood based-pulps or other non-wood derived fiber sources. The pulp may be a primary fibrous material or a secondary fibrous material (“recycled”). Sources of pulp fibers include, by way of example, woods, such as softwoods and hardwoods; straws and grasses, such as rice, esparto, wheat, rye, and sabai; canes and reeds, such as bagasse; bamboos; woody stalks, such as jute, flax, kenaf, and cannabis; bast, such as linen and ramie; leaves, such as abaca and sisal; and seeds, such as cotton and cotton liners. Softwoods and hardwoods are the more commonly used sources of cellulose fibers. Examples of softwoods include, by way of illustration only longleaf pine, shortleaf pine, loblolly pine, slash pine, Southern pipe, black spruce, white spruce, jack pine, balsam fir, douglas fir, western hemlock, redwood, and red cedar. Examples of hardwoods include, again by way of illustration only, aspen, birch, beech, oak, maple, eucalyptus, and gum. Specific examples of such pulp fibers include Northern Bleached Softwood Kraft (NBSK) pulps. An example of this pulp is LL-19 (formerly produced by Neenah Paper, Inc.) and INTERNATIONAL PINE® from International Paper Company. Other cellulosic fibers that may be used the present invention include eucalyptus fibers, such as Primacell Eucalyptus, available from Klabin Riocell, and other Northern Bleached Hardwood Kraft Pulps (NBHK) pulps. An example is LL-16 (formerly produced by Neenah Paper, Inc.), St. Croix hardwood available from Georgia-Pacific Corporation, and Leaf River hardwood available from Georgia-Pacific Corporation.

The pulp fibers may generally be chemical or mechanical pulp. Chemical pulp refers to fibrous materials from which most non-cellulose components are removed by chemical pulping without substantial mechanical post-treatment. Sulfite or sulfate (Kraft) chemical processes, for example, involve the dissolution of the lignin and hemi-cellulose components from the wood to varying degrees depending on the desired application. Mechanical pulp refers to fibrous materials made of wood processed by mechanical methods. Mechanical pulp is subdivided into the purely mechanical pulps (e.g., groundwood pulp and refiner mechanical pulp) and mechanical pulps subjected to chemical pretreatment (e.g., chemimechanical pulp or chemithermomechanical pulp). Synthetic cellulose-containing fibers may also be used, such as cellulosic esters, cellulosic ethers, cellulosic nitrates, cellulosic acetates, cellulosic acetate butyrates, ethyl cellulose, regenerated celluloses (e.g., viscose, rayon, etc.).

Although not required, the cellulosic fibrous material used to form the medical packaging substrate of the present invention is typically a chemical pulp. Examples of such chemical pulps include, for instance, sulfite pulps, Kraft pulps (sulfate), soda pulps (cooked with sodium hydroxide), pulps from high-pressure cooking with organic solvents, and pulps from modified processes. Sulfite and Kraft pulps differ considerably in terms of their fibrous material properties. The individual fiber strengths of sulfite pulps are usually much lower than those of Kraft pulps. The mean pore width of the swollen fibers is also greater in sulfite pulps and the density of the cell wall is lower compared to Kraft pulps, which simultaneously means that the cell-wall volume is greater in sulfite pulps. Due to their higher strength, lower pore width, and higher density, Kraft pulps are typically employed in the present invention.

While the present invention has applicability to any of the above chemical pulping processes, it is particularly useful with the Kraft process and, as such, the Kraft process is described in more detail below. Initially, suitable trees are harvested, debarked and then chipped into suitable size flakes or chips. These wood chips are sorted with the small and the large chips being removed. The remaining suitable wood chips are then charged to a digester (vessel or tank for holding the chips and an aqueous digesting composition and which can be operated in either a batch or continuous mode). In a batch type digester, wood chips and a mixture of “weak black liquor”, the spent liquor from a previous digester cook, and “white liquor”, a solution of sodium hydroxide and sodium sulfide, which is either fresh or from the chemical recovery plant, is pumped into the digester. In the cooking process, lignin, which binds the wood fiber together, is dissolved in the white liquor forming pulp and black liquor. The digester is sealed and heated to a suitable cook temperature (e.g. up to about 180° C.) under high pressure. After an allotted cooking time at a particular temperature and pressure (H-factor) in the digester, its contents (pulp and black liquor) are transferred to a holding tank. The pulp in the holding tank is transferred to the brown stock washers while the liquid (black liquor formed in the digester) is sent to the black liquor recovery area. The black liquor is evaporated to a high solids content, usually 60-80% solids. Once cooked, the pulp is typically subjected to a bleaching process to delignify the material. Chlorine, chlorine dioxide, sodium hypochlorite, hydrogen peroxide, oxygen, ozone, and mixtures thereof, are employed in most conventional bleaching processes. Ozone is a particularly effective bleaching technique, and may be used to perform low consistency, medium consistency, or high consistency bleaching. Ozone bleaching is normally performed an acidic pH level (less than 7) to optimize delignification effectiveness.

Once cooked and optionally bleached, the raw cellulosic fibrous material is supplied for web formation in accordance with the present invention. Different cellulosic fibers may be selected to provide different attributes. The choice of fiber sources depends in part on the final application of the web. For example, softwood fibers may be included in the web to increase tensile strength. Hardwood fibers may be selected for their ability to improve formation or uniformity in distribution of the fibers. In one embodiment, the fibrous web may contain from about 30% to about 75% eucalyptus fibers based on total dry weight of the fibers, and in some embodiments, from about 50% to about 75% eucalyptus fibers based on total fiber dry weight. Likewise, the fibrous web may contain from about 25% to about 70% eucalyptus fibers based on total dry weight of the fibers, and in some embodiments, from about 25% to about 50% softwood fibers based on total fiber dry weight.

If desired, synthetic fibers may also be used in conjunction with the cellulosic fibers to increase the tear resistance of the fibrous web. Examples of such synthetic fibers may include, for instance, polyolefins (e.g., polyethylene, polypropylene, polybutylene, etc.); polytetrafluoroethylene; polyesters (e.g., polyethylene terephthalate); polyvinyl acetate; polyvinyl chloride acetate; polyvinyl butyral; acrylic resins (e.g., polyacrylate, polymethylacrylate, polymethylmethacrylate, etc.); polyamides (e.g., nylon 6, nylon 6/6, nylon 4/6, nylon 11, nylon 12, nylon 6/10, and nylon 12/12); polyvinyl chloride; polyvinylidene chloride; polystyrene; polyvinyl alcohol; polyurethanes; polylactic acid; and so forth. The synthetic fibers may be monocomponent or multicomponent fibers. One example of a multicomponent fiber is comprised of two fibers having differing characteristics combined into a single fiber, commonly called a biocomponent fiber. Bicomponent fibers generally have a core and sheath structure where the core polymer has a higher melting point than the sheath polymer. Other bicomponent fiber structures, however, may be utilized. For example, bicomponent fibers may be formed with the two components residing in various side-by-side relationships as well as concentric and eccentric core and sheath configurations. One particular example of a suitable bicomponent fiber is available from KoSa under the designation CELBOND® T-255. CELBOND® T-255 is a synthetic polyester/polyethylene bicomponent fiber capable of adhering to cellulosic fibers when its outer sheath is melted at a temperature of approximately 128° C. When utilized, the synthetic fibers typically constitute from about 0.1% to about 30%, in some embodiments from about 0.1% to about 20%, and in some embodiments, from about 0.1% to about 10% of the dry weight of the web.

Particularly when natural fibers are employed, the fibrous material is generally placed in a conventional papermaking fiber stock prep beater or pulper containing a liquid, such as water. The fibrous material stock is typically kept in continued agitation such that it forms a suspension. If desired, the fibrous material may also be subjected to one or more refinement steps to provide a variety of benefits, including improvement of the bacterial filtration properties of the fibrous web. Refinement results in an increase in the amount of intimate contact of the fiber surfaces and may be performed using devices well known in the art, such as a disc refiner, a double disc refiner, a Jordan refiner, a Claflin refiner, or a Valley-type refiner. Various suitable refinement techniques are described, for example, in U.S. Pat. No. 5,573,640 to Frederick, et al., which is incorporated herein in its entirety by reference thereto for all purposes. The level of fiber degradation imparted by refinement may be characterized as “Canadian Standard Freeness” (CSF) (TAPPI Test Methods T-227 OM-94). For example, 800 CSF represents a relatively low amount of degradation, while 400 CSF represents a relatively high amount of degradation. In most embodiments of the present invention, the fibers are refined to about 400 to about 800 CSF, and in some embodiments, from about 600 CSF to about 750 CSF.

The resulting fibrous suspension may then be diluted and readied for formation into a fibrous web using conventional papermaking techniques. For example, the web may be formed by distributing the suspension onto a forming surface (e.g., wire) and then removing water from the distributed suspension to form the web. This process may involve transferring the suspension to a dump chest, machine chest, clean stock chest, low density cleaner, headbox, etc., as is well known in the art. Upon formation, the fibrous web may then be dried using any known technique, such as by using convection ovens, radiant heat, infrared radiation, forced air ovens, and heated rolls or cans. Drying may also be performed by air drying without the addition of thermal energy. If desired, the fibers may be treated with the pH modifier at any stage of the papermaking process.

Regardless of the manner in which the web is formed, the cellulosic fibrous material may be treated with a pH modifier so that its pH is about 7.0 or more, in some embodiments from about 7.0 to about 9.0, and in some embodiments, from about 7.5 to about 8.0. Through treatment with the pH modifier, the resulting medical packaging substrate may also have a pH of greater than about 7.0, in some embodiments greater than about 7.1, and in some embodiments, greater than about 7.2. Suitable pH modifiers may include, but are not limited to, ammonia; mono-, di-, and tri-alkyl amines; mono-, di-, and tri-alkanolamines; alkali metal and alkaline earth metal hydroxides; alkali metal and alkaline earth metal silicates; alkali metal and alkaline earth metal carbonates; and mixtures thereof. Specific examples of pH modifiers are sodium carbonate (“soda ash”) and sodium bicarbonate; ammonia; sodium, potassium, and lithium hydroxide; sodium, potassium, and lithium meta silicates; monoethanolamine; triethylamine; isopropanolamine; diethanolamine; and triethanolamine. Although the amount of the pH modifier employed may vary, it is typically present in an amount of from about 0.001 wt. % to about 5 wt. %, in some embodiments from about 0.05 wt. % to about 1 wt. %, and in some embodiments, from about 0.1 wt. % to about 0.5 wt. % based on the dry weight of the fibers.

The present inventor has discovered that treatment with the pH modifier helps minimize any odor produced by the cellulosic fibrous material during ozone sterilization. Without intending to be limited by theory, it is believed that ozone attack causes cellulosic fibers to release odorous compounds, such as mercaptans (e.g., ethyl mercaptan), ammonia, amines (e.g., trimethylamine (TMA), triethylamine (TEA), etc.), sulfides (e.g., hydrogen sulfide, dimethyl disulfide (DMDS), etc.), ketones (e.g., 2-butanone, 2-pentanone, 4-heptanone, etc.) carboxylic acids (e.g., isovaleric acid, acetic acid, propionic acid, etc.), aldehydes, terpenoids, hexanol, heptanal, pyridine, and so forth. Through the use of a higher pH level, however, such reactions may be inhibited. In addition, certain pH modifiers (e.g., carbonates and bicarbonates) may also act as free radical scavengers for odorous compounds, such as aldehydes and/or carboxylic acids.

The treatment with the pH modifier may occur at any stage of the papermaking process, including prior to formation of the web, during web formation, and/or after web formation. In one embodiment, for instance, the pH modifier is simply added to the fiber suspension in the pulper. In addition, the fibrous web may be saturated with the pH modifier after it is formed. Any known saturation technique may be employed, such as brushing, flooded nip saturation, doctor blading, spraying, and direct and offset gravure coating. For example, the web may be exposed to an excess of the solution and then squeezed. The squeezing of excess pH modifier from the web may be accomplished by passing the web between rollers. If desired, the excess pH modifier may be returned to the supply for further use. After squeezing out excess material, the saturated web may then be dried.

In addition to pH modifiers, other additives may also be applied to the fibers. For example, wet-strength agents may be used to improve the strength properties of the web during formation. The wet-strength agent may be present in an amount from about 0.001 wt. % to about 5 wt. %, in some embodiments from about 0.01 wt. % to about 2 wt. %, and in some embodiments, from about 0.1 wt. % to about 1 wt. %, based on the dry weight of the fibers. Wet strength agents are typically water soluble, cationic oligomeric or polymeric resins that are capable of bonding with the cellulosic fibers. Although various wet-strength agents are known in the papermaking art, the present inventor has discovered that certain types of wet strength agents provide superior odor reduction when the resulting web is subjected to ozone sterilization. For example, some wet-strength agents found to produce minimal odor upon ozone sterilization are polyamine-epichlorohydrin, polyamide epichlorohydrin or polyamide-amine epichlorohydrin resins (collectively “PAE” resins). Examples of these materials are described in U.S. Pat. Nos. 3,700,623 to Keim and 3,772,076 to Keim, which are incorporated herein in their entirety by reference thereto for all purposes. Suitable PAE resins are available from Hercules, Inc. of Wilmington, Del. under the designation “KYMENE®” (e.g., KYMENE® 557H or 557 LX). KYMENE® 557 LX, for example, is a polyamide epicholorohydrin polymer that contains both cationic sites, which may form ionic bonds with anionic groups on the pulp fibers, and azetidinium groups, which may form covalent bonds with carboxyl groups on the pulp fibers and crosslink with the polymer backbone when cured. Other suitable polyamide-epichlorohydrin resins are described in U.S. Pat. Nos. 3,885,158 to Petrovich; 3,899,388 to Petrovich; 4,129,528 to Petrovich; 4,147,586 to Petrovich; and 4,222,921 to van Eanam, which are incorporated herein in their entirety by reference thereto for all purposes.

Of course, other wet strength agents may also be employed in certain embodiments of the present invention. For example, other suitable wet strength agents may include dialdehyde starch, polyethylene imine, mannogalactan gum, glyoxal, and dialdehyde mannogalactan. Particularly useful wet-strength agents are water-soluble polyacrylamide resins available from Cytec Industries, Inc. of West Patterson, N.J. under the designation PAREZ® (e.g., PAREZ® 631NC). The PAREZ® resins are formed from a polyacrylamide-glyoxal polymer that contains cationic hemiacetal sites. These sites may form ionic bonds with carboxyl or hydroxyl groups present on the cellulosic fibers to provide increased strength to the web. Because the hemiacetal groups are readily hydrolyzed, the wet strength provided by the resins is primarily temporary. Such resins are believed to be described in U.S. Pat. Nos. 3,556,932 to Coscia, et al. and 3,556,933 to Williams, et al., which are incorporated herein in their entirety by reference thereto for all purposes.

In accordance with the present invention, a binder composition is also applied to the fibers, before and/or after web formation, to further improve the strength properties of the web. Typically, the binder composition includes a latex polymers, such as polyacrylates, including polymethacrylates, poly(acrylic acid), poly(methacrylic acid), and copolymers of the various acrylate and methacrylate esters and the free acids; styrene-butadiene copolymers; ethylene-vinyl acetate copolymers; nitrile rubbers or acrylonitrile-butadiene copolymers; poly(vinyl chloride); poly(vinyl acetate); ethylene-acrylate copolymers; vinyl acetate-acrylate copolymers; neoprene rubbers or trans-1,4-polychloroprenes; cis-1,4-polyisoprenes; butadiene rubbers or cis- and trans-1,4-polybutadienes; and ethylene-propylene copolymers. Although any latex polymer may generally be employed, the present inventor has nevertheless discovered that certain latex polymers are particularly effective in minimizing odor upon ozone sterilization. For example, many conventional latex polymers are reacted with N-methylol acrylamide, N-(n-butoxy methyl) acrylamide, N-(iso-butoxy methyl) acrylamide, N-methylol methacrylamide, and other similar crosslinking agents. Unfortunately, these monomers undergo a condensation reaction on crosslinking that evolves formaldehyde (CH₂O). Upon ozone sterilization, the release of formaldehyde further increases the odor produced. Thus, it is often desired to select a latex polymer that contains carboxyl functional groups, such as carboxylated (carboxy-containing) polyacrylates, carboxylated nitrile-butadiene copolymers, carboxylated styrene-butadiene copolymers, carboxylated ethylene-vinylacetate copolymers, and polyurethanes. Specific examples of suitable carboxylated, formaldehyde-free latex polymers are polyacrylate binders available under the designations HYCAR® 26469, 26552, and 26703 from Noveon, Inc. of Cleveland, Ohio. The carboxylated latex polymer may be self-crosslinking. Alternatively, a crosslinking agent may be employed that is reactive to the carboxyl groups without releasing formaldehyde. One example of such a crosslinking agent is an aziridine oligomer having at least two aziridine functional groups, such as XAMA®-7 (Noveon, Inc. of Cleveland, Ohio) and Chemitite PZ-33 (Nippon Shokubai Co. of Osaka, Japan).

In addition to a latex polymer, the binder composition may also contain a heat-sealable polymer to help improve the peel strength of the resulting medical package during use. Examples of such heat-sealable polymers include, but are not limited to, homopolymers and heteropolymers of lower alkenes, e.g., ethylene and/or propylene. Specific examples of such heat-sealable polymers are polyethylene, polypropylene, ethylene acrylic acid, and ethylene vinyl acetate. One particularly desirable heat-sealable polymer is ethylene acrylic acid, such as commercially available under the name “Michem® Prime 4983R” from Michelman, Inc. Michem® Prime 4983R is a dispersion of Dow PRIMACOR® 59801 (copolymer of ethylene and acrylic acid that has an ethylene content of approximately 80%). Other suitable heat-sealable polymers may be described in U.S. Pat. No. 6,887,537 to Bean, et al., which is incorporated herein in its entirety by reference thereto for all purposes. When employed, heat-sealable polymers may constitute from about 35 wt. % to about 85 wt. %, in some embodiments, from about 40 wt. % to about 70 wt. %, and in some embodiments, from about 50 wt. % to about 60 wt. % of the binder composition. Likewise, latex polymers may constitute from about 25 wt. % to about 75 wt. %, in some embodiments from about 30 wt. % to about 60 wt. %, and in some embodiments, from about 40 wt. % to about 50 wt. % of the binder composition.

The binder composition may be applied to the cellulosic fibrous material before, during, and/or after web formation using any technique known in the art. Preferably, the binder composition is impregnated into the fibrous web in a manner such as described above. Other suitable techniques for impregnating a web with a binder composition are described in U.S. Pat. No. 5,595,828 to Weber and U.S. Patent Application Publication No. 2002/0168508 to Reed, et al., which are incorporated herein in their entirety by reference thereto for all purposes. The amount of the binder composition applied may vary depending on the desired properties of the web, such as the desired permeability. Typically, the binder composition is present at an add-on level of from about 10% to about 90%, in some embodiments from about 20% to about 70%, and in some embodiments, from about 30% to about 60%. The add-on level is calculated, on a dry weight basis, by dividing the dry weight of binder composition applied by the dry weight of the web before treatment, and multiplying the result by 100.

If desired, the fibrous web may also be applied with an adhesive coating to enhance peel strength, decrease permeability, and/or increase the bacteria barrier. The coating may include any known adhesive, including pressure-sensitive or hot-melt adhesives.

In addition to the ingredients set forth above, various other additives may also be employed in the fibrous web. The additives may be applied directly the web or fibers, in conjunction with the binder composition or adhesive coating, or as a separate coating. By way of example, suitable additives may include antifoaming agents, pigments, processing aids, and dispersing agents. Examples of antifoaming agents include, but are not limited to, products such as NALCO® 7518 available from Nalco Chemical Company or DOW Corning® Antifoam available from Dow Corning Corporation. Dispersing agents or surfactants include, but are not limited to, products such as TAMOL® 731A available from Rohm & Haas Co., PLURONIC® F108 available from BASF Corporation, SMA® 1440 Resin available from ATOFINA Chemicals, Inc., and TERGITOL® 15S available from Union Carbide Corp. Examples of processing aids may include, but are not limited to, products such as NOPCOTE® DC-100A available from Geo Specialty Chemicals, Inc., SCRIPSET® 540 available from Solutia, Inc. and AQUAPEL® 752 available from Hercules Incorporated. Examples of pigments used to increase opacity include but are not limited to, titanium dioxide such as TI-PURE® Rutile Titanium Dioxide available from E.I. Du Pont De Nemours & Co. and kaolin pigments, which are available from a variety of manufacturers. A wide range of pigments and dyes may also be added to impart color to the saturated sheet. The foregoing list of categories of additives and examples of categories is provided by way of example and is not intended to be exhaustive.

Regardless of the particular manner in which it is formed, the fibrous web of the present invention possesses certain characteristics that facilitate its use in ozone sterilization processes. For example, the permeability of the web (with optional coatings) is generally high enough to allow for the flow of ozone gas during sterilization, but not so high as to significantly increase the ability of bacteria or other pathogens to penetrate through the web. One indicator of the permeability of a web is “Gurley porosity”, which is determined in accordance with TAPPI Test Method No. T 460 om-96 (1996). High Gurley porosity values correspond to low web permeability, and low Gurley porosity values likewise correspond to high web permeability. When used as a medical packaging substrate, the web of the present invention typically has a Gurley porosity of from about 10 to about 120 seconds per 100 cubic centimeters, in some embodiments from about 20 to about 80 seconds per 100 cubic centimeters, and in some embodiments, from about 30 to about 60 seconds per 100 cubic centimeters. As would be readily understood to those skilled in the art, the porosity of the web may be achieved through modification of a variety of parameters, including the type and amount of the binder composition, the type and weight of the fibrous web, and so forth.

Further, the medical packaging substrate of the present invention also exhibits good barrier efficacy to bacteria, as expressed by percent bacterial filtration efficiency (“BFE”). The percent BFE generally represents the ability of a sample to act as a barrier to microorganisms and has an upper limit of 100%, which indicates that 100% of the microorganisms were intercepted by the test material. Typically, the percent BFE of the medical packaging substrate of the present invention is at least about 95%, in some embodiments at least about 97%, and in some embodiments, at least about 99%. Another parameter that is indicative of the barrier efficacy of the medical packaging substrate of the present invention is the log reduction value (“LRV”). LRV is the difference, measured in log scale, between the number of colony forming units (“CFU”) on a control media and the number of CFU on a test media. The range of measurable LRV is generally between 0 to 5, where higher numbers indicate greater barrier efficacy. The number of colony forming units may be measured in accordance with ASTM F 1608-95. Typically, the medical packaging substrate of the present invention exhibits a LRV of at least about 3, in some embodiments at least about 4, and in some embodiments, about 5. A more detailed description of the manner in which % BFE and LRV are determined is provided in U.S. Pat. No. 6,887,537 to Bean, et al.

The fibrous web of the present invention may be utilized as a sterilization package in any manner known to those skilled in the art. For example, the web may be sealed to a base component using a heat seal device that applies heat to the edges or surfaces of the web and base component (optionally, in conjunction with an adhesive) to form a pouch, rigid container (e.g., tub or tray), etc. Typical materials used for the base component include, but are not limited to, nylon, polyester, polypropylene, polyethylene (e.g., low density, linear low density, ultra low density and high density polyethylene), and polystyrene. Examples of such packages are described, for instance, in U.S. Pat. Nos. 3,991,881 to Augurt; 4,183,431 to Schmidt, et al.; 5,217,772 to Brown, et al.; and 5,418,022 to Anderson, et al., which are incorporated herein in their entirety by reference thereto for all purposes.

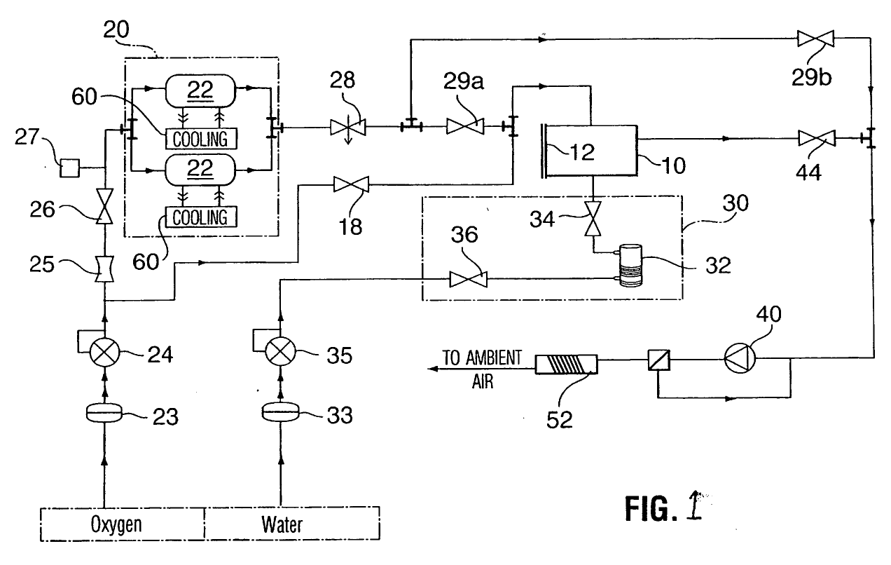

The contents of the packaging may generally vary as is well known in the art and may include, for instance, surgical devices, implants, tubing, valves, gauzing, syringes, protective clothing (e.g., surgical gowns, drapes and gloves), or any other sterilizable item. Once the packaging is provided with the desired contents, it is then subjected to ozone sterilization. Various ozone sterilization techniques may be utilized in the present invention. For examples, several suitable ozone sterilization techniques are described in U.S. Pat. Nos. 5,069,880 to Karlson; 5,868,999 to Karlson; and 6,365,103 to Fournier, as well as U.S. Patent Application Publication No. 2002/0085950 to Robitaille, et al., all of which are incorporated herein in their entirety by reference thereto for all purposes. In this regard, one particular embodiment of an ozone sterilizing apparatus that may be employed in conjunction with the present invention is shown in FIG. 1. As shown, the apparatus includes a sterilization chamber 10 that may be sealed to contain a vacuum. This is achieved with an access door 12, which may be selectively opened for access into the chamber and which seals the chamber in the closed condition. The apparatus further includes an ozone generating unit 20 for supplying an ozone-containing gas to the sterilization chamber 10, a humidifier arrangement 30 for supplying water vapor to the sterilization chamber, and a vacuum pump 40 (e.g., Trivac® D25BCS PFPE from Leybold). The vacuum pump 40 is used to supply a sufficient vacuum to the sterilization chamber 10 to increase the penetration of the sterilizing gas and to generate water vapor at a temperature below the temperature inside the sterilization chamber 10. The vacuum pump 40 may also lower the boiling point of water in the chamber 10 below the chamber temperature. For example, the vacuum pump 40 may produce a vacuum pressure of about 0.1 millibar.

The apparatus also includes an ozone-converting unit 52 to which the ozone-containing gas is fed either after passage through the sterilization chamber 10 or directly from the ozone-generating unit 20 through valve 29b. The ozone-converting unit 52 is connected in series after the vacuum pump 40 to inhibit the escape of ozone gas. The ozone-converting unit 52 contains a catalyst that destroys the ozone on contact and reverts it back into oxygen. An example of such an ozone converting catalyst is DEST 25, which is available from TSO₃. Other catalysts of this type and their manufacture are well known to a person skilled in the art of ozone generators and need not be described in detail herein. Furthermore, other methods for destroying the ozone contained in the sterilization gas will be readily apparent to a person skilled in the art. For example, the gas may be heated for a preselected time to a temperature at which the ozone decomposition is accelerated (e.g., 300° C.).

The humidifier arrangement 30 includes a humidifier chamber 32 (HUM 0.5, manufacturer TSO₃) sealed to ambient and connected to the sterilization chamber 10 through a conduit and a vapor intake valve 34. The humidifier chamber 32 is equipped with a level control (not shown) to ensure a sufficiently high water level. Water is supplied to the humidifier chamber 32 through a filter 33, a pressure regulator 35, and input valve 36. The water vapor produced in the humidifier chamber 32 enters the sterilization chamber 10 by way of a vapor intake valve 34.

The ozone-generating unit 20 includes a pair of ozone generators 22 (OZ, model 14a, manufacturer TSO₃) of the corona discharge type, which are cooled to decrease the ozone decomposition rate. To improve the lethality rate of the ozone sterilization process, the concentration of ozone supplied to the sterilization chamber is typically from about 45 to 100 about milligrams of ozone per liter of gas, and preferably from about 60 to about 75 milligrams of ozone per liter of gas. At these concentrations, the ozone generation is associated with a relatively high energy loss in the form of heat. Generally, about 95% of the supplied electrical energy is converted into heat and only 5% is used to produce ozone. Because heat accelerates the inverse transformation of ozone into oxygen, it is removed as quickly as possible by cooling the ozone generators 22. Thus, a cooling system 60 is employed to keep the generators 22 at a relatively low temperature (e.g., 3 to 6° C.) so that the ozone-containing gas generated is at a temperature of from about 20° C. to about 35° C. This keeps ozone decomposition to a minimum to enhance the efficiency of the sterilization process.

The ozone-generating unit is typically supplied with medical quality oxygen. The apparatus may be connected to a wall oxygen outlet common in hospitals or to an oxygen cylinder or to any other source capable of supplying the required quality and flow. The supply of oxygen to the generators 22 takes place across a filter 23, a pressure regulator 24, a flow meter 25 and an oxygen shut off valve 26. The generators 22 are protected against oxygen overpressure by a safety pressure switch 27. The ozone-oxygen mixture generated by the generators 22 is directed to the sterilization chamber 10 by a regulator valve 28 and a mixture supply solenoid valve 29a. The mixture may also be directly supplied to the ozone-converting unit 52 by way of a bypass solenoid valve 29b. In one embodiment, the pressure regulator 24 controls the oxygen input at a flow rate of from about 1.5 to 2 liters per minute. However, it will be readily apparent to the skilled person that other flow rates may be used depending on the make and model of the ozone generators 22 and the size of the sterilization chamber 10.

One embodiment of a method for using the sterilization apparatus of FIG. 1 will now be described in more detail. Initially, medical instruments to be sterilized are sealed in the medical packaging of the present invention and then placed into the sterilization chamber 10. The sterilization chamber 10 is then sealed and a vacuum pressure is supplied. Before sterilization begins, the humidifier chamber 32 is filled with water to an adequate level, which is sufficient to satisfy the requirements for the sterilization cycle. This is accomplished by temporarily opening the water-input valve 36. Valve 36 remains closed for the remainder of the sterilization cycle. In the first phase of the sterilization cycle, oxygen intake valve 18, oxygen shut-off valve 26, mixture supply valve 29a, and mixture bypass valve 29b are closed and vapor intake valve 34, chamber drainage valve 44, and a bypass valve are opened. The sterilization chamber 10 is evacuated to a vacuum pressure (e.g., about 0.1 millibar). Water vapor inlet valve 34 closes when the absolute pressure in the sterilization chamber 10 falls below a certain level (e.g., about 60 millibars). Once a desired pressure is achieved, the chamber drainage valve 44 closes and the vapor intake 34 opens to lower the pressure in the humidifier chamber 32 to the vacuum pressure in the sterilization chamber 10. This forces the water in the humidifier chamber 32 to evaporate and to enter the sterilization chamber 10. Shortly before the end of the humidification period (e.g., about 2 to 6 minutes), the ozone generators 22 are activated. The oxygen/ozone mixture exiting the ozone generators 22 may be controlled by a regulator valve 28 to a flow rate of, for example, from about 1.5 to about 2 liters per minute. The generators 22 may also be started at the same time as the humidification period using a shut-off valve 26 and mixture bypass valve 29b. The shut-off valve 26 opens to let oxygen enter the generators 22, and the resulting ozone-oxygen mixture is then guided directly into the ozone-converting unit 52 through the mixture bypass valve 29b.

After the humidification period (e.g., about 30 minutes), the oxygen-ozone mixture is guided into the sterilization chamber 10 by opening the mixture supply valve 29a and closing the mixture bypass valve 29b. The oxygen-ozone mixture enters the chamber 10 until the desired ozone concentration (e.g., 85 milligrams of ozone per liter of gas) is achieved. The time required for this step depends on the flow rate and concentration of the ozone gas in the mixture (e.g., about 10% to 12% by weight of the gas). At this point, the mixture supply valve 29a is closed to seal off the sterilization chamber 10 and to maintain the humidified ozone/oxygen gas mixture under vacuum. Beneficially, the oxygen-ozone mixture may be injected into sterilization chamber 10 at ambient temperature, such as from about 20° C. to about 35° C.

Once the sterilization chamber 10 is filled with the sterilization gas, the generators 22 are stopped, the oxygen shut-off valve 26 is closed, and the ozone is maintained in contact with the article to be sterilized. The length of this sterilization period varies with the volume of the sterilization chamber 10 (e.g., about 15 minutes for a volume of 125 liters). At this stage, the sterilization chamber 10 is still under the effect of a partial vacuum (e.g., about 500 to 525 millibars). In an optional second step, the pressure level is raised (e.g., to about 900 millibars) using oxygen as a filling gas. After the sterilization period, the vacuum is reapplied and the humidification phase is recommenced, followed by the renewed injection of an oxygen/ozone sterilization gas mixture. The cycle of applying a vacuum, injecting sterilization gas, and humidifying and sterilization period, may be repeated to achieve desired level of sterilization.

Upon completion, the remaining ozone and humidity is removed from the sterilization chamber 10 during a ventilation phase. Specifically, the ventilation phase begins by opening the chamber drainage valve 44 and reducing the vacuum pressure (e.g., to about 13 millibars). The vapor intake valve 34 closes when the pressure reaches a certain point (e.g., about 60 millibars) to evacuate the remaining ozone. Once the desired vacuum pressure is obtained, the drainage valve 44 closes and the oxygen intake valve 18 opens, admitting oxygen into the sterilization chamber 10. Upon reaching atmospheric pressure, the oxygen intake valve 18 is closed, the sterilization chamber drainage valve 44 is opened, and vacuum pressure is reapplied. The ventilation cycle may then be repeated one or more times. After reaching atmospheric pressure in the final cycle, the door 12 of the sterilization chamber 10 is activated to permit access to the sterilized contents.

After sterilization, the medical packaging substrate of the present invention may exhibit a much lower reduction in its tensile characteristics (e.g., strength and elongation) than would normally be expected for cellulosic materials subjected to ozone degradation. For example, the medical packaging substrate may exhibit a loss in tensile strength in the machine direction (“MD”) and/or cross-machine direction (“CD”) of no more than about 40%, in some embodiments, about 35%, and in some embodiments, about 30%. The percent loss in tensile strength is determined by subtracting the tensile strength after sterilization from the strength prior to sterilization, and then dividing by the tensile strength after sterilization. Typically, the MD tensile strength is at least about 4, in some embodiments at least about 5, and in some embodiments, at least about 6 kilograms per 15 millimeters (“kg/15 mm”) after ozone sterilization. The CD tensile strength is typically at least about 2, in some embodiments at least about 3, and in some embodiments, at least about 4 kg/15 mm after ozone sterilization. Likewise, the medical packaging substrate may also exhibit a loss in stretch or elongation in the MD and/or CD directions of no more than about 50%, in some embodiments, about 45%, and in some embodiments, about 40%. The percent loss in elongation is determined by subtracting the elongation after sterilization from the elongation prior to sterilization, and then dividing by the elongation after sterilization. Typically, the MD elongation is about 1% or more, and in some embodiments, about 2% or more after ozone sterilization. The CD elongation is typically about 4% or more, and in some embodiments, about 5% or more after ozone sterilization.

Moreover, when sealed to a base component to form a package, the seal may maintain sufficient strength to ensure that stresses resulting from package handling after assembly will not cause the seal to open before the desired time and will remain impervious to pathogens. This strength is commonly expressed as the force required to separate the two sealed layers (i.e., “peel strength”). The peel strength may be determined in accordance with ASTM F904-98 using a tensile tester (e.g., Instron Model 5500R tensile tester). For example, the package may be positioned so that the medical packaging substrate is located adjacent to one jaw and the base component (e.g., film) is located adjacent to the other jaw. The sample may then be pulled apart at 90° until the sample breaks. The peel strength is the force (in kilograms) required to break the sample. A package formed according to the present invention maintains a good peel strength even after ozone sterilization. For example, the peel strength of the package after ozone sterilization may be at least about 0.70 pound per square inch, in some embodiments from about 1.0 to about 2.5 pounds per square inch, and in some embodiments, from about 1.2 to about 2.0 pounds per square inch. At such peel strengths, the package will tear at the seal line only when opened.

The present invention may be better understood with reference to the following examples.

### Test Methods (as Not Otherwise Described Above)

Strength Properties:

Machine direction (“MD”) and cross-machine direction (“CD”) tensile strengths were determined using an Instron Model 5500R tensile tester. The test samples were parallel-edged strips having a 15-mm wide cut on a Tensile Strip Cutter. Strips were cut to a length of 7 inches. Prior to testing, the samples were conditioned at 73° F. and 50% relative humidity for a minimum of 4 hours. Tensile strength was reported as the force (in kilograms) required to break the sample in either the machine or cross direction. The percent stretch was the total elongation of the sample at the automatic breakpoint and was reported as a percent of the original sample length. In the calculation for stretch, the automatic “break” point was determined in one of two ways depending upon the shape of the testing curve close to the actual break point. Specifically, if the curve dropped off sharply toward the actual specimen break, the automatic “break” point was found on the shoulder of the curve before it dropped off toward the x-axis. For this criterion to be used, the drop-off line must be close to vertical. If the curve dropped off less abruptly, the automatic “break” point was determined where the slope of a line, tangent to the curve, was at a minimum.

Gas Chromatography and Mass Spectrometery:

Two-gram control samples were put into a 60-milliliter vial sealed with Teflon-coated septa. The samples were tested by poking a small hole in the aluminum foil wrapping the samples and inserting a 85-micrometer Carboxen/polydimethylsilicone “Solid Phase Microextraction” (SPME) assembly for about 30 minutes to collect the volatiles for analysis (Supelco catalog No. 57330-U fiber holder and 57334-U 85 Carboxen/polydimethylsilicone on a StableFlex fiber). The SPME extracts were analyzed by gas chromatography and mass spectrometery (“GC/MS”) using a system available from Agilent Technologies, Inc. under the name “5973N.” Helium was used as the carrier gas. A DB-5MS column was used, which is available from J&W Scientific, Inc. of Folsom, Calif. The total ion chromatograms were determined and the peaks of the spectrum were matched to a corresponding compound.

pH:

The pH of a liquid furnish, dispersion, or solution was measured using a pH meter obtained from Corning (Model 1220). The pH of sample substrates was also measured as follows. The sample was cut into approximately 0.5-inch squares and weighed. The squares were placed into a clean bottle (4 to 6 ounces), into which 70 milliliters of distilled, deionized water was added. The bottle was closed and shaken, and then allowed to stand at room temperature for 1 hour. The bottle was again shaken two or three times to ensure complete wetting of the substrate. After standing for at least 1 hour, the pH of the water was measured with the pH meter referenced above.

### EXAMPLE 1

The ability to form a medical packaging substrate in accordance with the present invention was demonstrated. The substrate was formed from a blend of pulp fibers containing 55.6 wt. % LL-19 and 44.4 wt. % LL-16. Kymene® 557LX (Hercules, Inc.) was also added to the pulp furnish. LL-19 is a bleached Northern softwood pulp and LL-16 is a bleached Northern hardwood pulp. Four (4) different samples were then formed.

For Sample 1, 9 ounces of soda ash were added to the pulp furnish in the pulper per 2430 pounds of pulp so that the pH of the pulp furnish was approximately 7.0.

For Sample 2, 22 ounces of soda ash were added to the machine chest per 2430 pounds of pulp so that the pH of the pulp furnish was approximately 7.35. The resulting web was then saturated on-line with a dispersion that contained 79.3 wt. % Hycar® 26469 (Noveon, Inc.), 19.8 wt. % of a pigment, and small quantities of ammonia and Nopcote DC-100A.

For Sample 3, the web was formed and then saturated off-line with an aqueous solution of soda ash (0.1 wt. %) having a pH of 10.5. Thereafter, the web was saturated with a dispersion that contained 79.3 wt. % Hycar® 26469 (Noveon, Inc.), 19.8 wt. % of a pigment dispersion and small quantities of Ammonia and Nopcote DC-100A.

Finally, for Sample 4, the web was formed and then saturated off-line with an aqueous solution of soda ash (0.3 wt. %) having a pH of 10.5. Thereafter, the web was saturated with a dispersion that contained 79.3 wt. % Hycar® 26469 (Noveon, Inc.), 19.8 wt. % of a pigment dispersion and small quantities of ammonia and Nopcote DC-100A.

Each of the saturated samples set forth above (Samples 2-4) were then subjected to ozone sterilizing using an ozone sterilization system available from TSO₃ (Model 125L). The sterilized samples were then tested for tensile strength, stretch and porosity effects. The physical properties are set forth below in Table 2.

TABLE 2
Physical Properties of Samples

| | | Tensile | | | | Loss of | | Loss of | | |
|---|---|---|---|---|---|---|---|---|---|---|
| | | Strength | | Stretch | | Strength | | stretch | | |
| | | (kg/15 mm) | | (%) | | (%) | | (%) | | Gurley |
| Sample | Condition | MD | CD | MD | CD | MD | CD | MD | CD | porosity |
| 2 | Before | 7.70 | 6.60 | 3.10 | 8.07 | — | — | — | | 30.4 |
| | Sterilization | | | | | | | | | |
| | After | 6.10 | 4.80 | 1.86 | 5.41 | 26.2 | 27.3 | 40.0 | 33.3 | 30.4 |
| | Sterilization | | | | | | | | | |
| 3 | Before | 8.20 | 5.90 | 2.35 | 8.16 | — | — | — | — | 18.4 |
| | Sterilization | | | | | | | | | |
| | After | 5.90 | 4.40 | 1.95 | 5.87 | 28.1 | 25.4 | 17.0 | 28.1 | 14.8 |
| | Sterilization | | | | | | | | | |
| 4 | Before | 8.4 | 5.9 | 2.74 | 8.41 | — | — | — | | 28.7 |
| | Sterilization | | | | | | | | | |
| | After | 6.40 | 4.40 | 2.01 | 6.18 | 23.8 | 25.4 | 26.6 | 26.5 | 24.4 |
| | Sterilization | | | | | | | | | |

As indicated, the ozone-sterilized samples made according to the present invention maintained good strength, stretch, and permeability characteristics. In addition, no odor was detected by a panel of individuals for the samples. The sheets samples were also tested for peel strength evaluation. The evaluation involved heat sealing a film to the paper substrate and then measuring the peel strength. Samples 3 and 4 exhibited more consistent peel strength than Sample 2.

The total ion chromatogram of Sample 3 was also determined and the peaks of the spectrum were matched to a corresponding compound. The results of the analysis are shown below in Table 3. This was compared to medical paper available from Neenah Paper, Inc. under the designation Impervon®. The Impervon® medical paper contains a Parez® 607L wet strength resin and is saturated with Hycar® 26703 and Michelman® 4983R. The results of the analysis are shown below in Table 4.

TABLE 3
Identity of the Largest Peaks of Total Ion Chromatogram
(Sample 3)

| Peak # | % of Total | Identity |
|---|---|---|
| 1 | 60.765 | Formic Acid |
| 2 | 24.674 | Acetic Acid |
| 3 | 0.742 | Propanoic Acid |
| 4 | 1.194 | Hexanal |
| 5 | 1.176 | Unknown |
| 6 | 0.826 | Unknown |
| 7 | 0.909 | Heptanal |
| 8 | 0.851 | Unknown |
| 9 | 0.352 | Benzadehyde (unknown) |
| 10 | 0.703 | Octanal |
| 11 | 0.656 | Dichlorobenzene |
| 12 | 1.222 | Unknown |
| 13 | 0.600 | Nonanal |
| 14 | 5.329 | Unknown |

TABLE 4
Identity of the Largest Peaks of Total Ion Chromatogram
(Commercial Sample)

| Peak # | % of Total | Identity |
|---|---|---|
| 1 | 4.853 | Air |
| 2 | 2.996 | Acetaldehyde |
| 3 | 11.300 | Propanal |
| 4 | 25.079 | Butanal + hexane |
| 5 | 4.408 | Pentanal |
| 6 | 1.047 | Butyl ethyl ketone |
| 7 | 15.219 | Toluene |
| 8 | 1.541 | Hexanal |
| 9 | 0.813 | Unknown |
| 10 | 1.358 | Unknown |
| 11 | 1.866 | Siloxane |
| 12 | 4.069 | Decane |
| 13 | 0.822 | Limonene |
| 14 | 1.631 | C4-benzene |
| 15 | 4.050 | Undecane |
| 16 | 3.451 | Siloxane |
| 17 | 1.697 | C4-benzene |
| 18 | 1.495 | C4-benzene |
| 19 | 2.147 | Azulene or napthalene |
| 20 | 0.831 | Siloxane |
| 21 | 0.986 | Methylnapthaklene |
| 22 | 1.712 | p-ethoxybenzoic acid |
| 23 | 4.053 | N-Cyclohexyl-2-pyrrlidone |

As indicated, Sample 3 exhibited prominent peaks for acetic acid and formic acid, but lacked significant amounts of the more odorous aldehyde and ketone compounds found in the commercially available sample.

### EXAMPLE 2

The substrate was formed from a blend of pulp fibers containing 52.7 wt. % LL-19 and 47.3 wt. % Aracruz Eucalyptus. Kymene® 557LX (Hercules, Inc.) was also added to the pulp furnish. LL-19 is a bleached Northern softwood pulp and Aracruz Eucalyptus is a bleached hardwood pulp. 8 ounces of soda ash were added to the pulp furnish in the pulper per 2332 pounds of pulp so that the pH of the pulp furnish was approximately 7.0. A web was formed and then saturated off-line with an aqueous solution of soda ash (0.1 wt %) having a pH of 10.5. Thereafter, the web was saturated with a dispersion that contained 37.3 wt. % Hycar® 26469 (Noveon, Inc.), 45.5 wt. % of Michelman® 4983R, 16.6% pigment, and a small quantity of Nopcote DC-100A. Pouches were then formed from the substrates that exhibited good peel strength and exhibited little odor upon sterilization. The pH of the paper was measured and determined to be 7.06. The total ion chromatogram was also determined and the peaks of the spectrum were matched to a corresponding compound. The results of the analysis are shown below in Table 5.

TABLE 5
Identity of the Largest Peaks of Total Ion Chromatogram

| Peak # | % of Total | Identity |
|---|---|---|
| 1 | 9.01 | carbon dioxide |
| 2 | 1.28 | ethanol + acetone |
| 3 | 26.26 | formic acid |
| 4 | 1.19 | butanal |
| 5 | 20.27 | acetic acid |
| 6 | 1.31 | pentanal |
| 7 | 4.46 | propanoic acid |
| 8 | 1.06 | toluene |
| 9 | 6.26 | butanoic acid |
| 10 | 3.40 | hexanal |
| 11 | 0.25 | Unknown |
| 12 | 2.11 | 1,2-ethanediol diformate |
| 13 | 3.00 | petanoic acid |
| 14 | 2.06 | heptanal |
| 15 | 1.25 | 4-methyl-3-pentenoic acid |
| 16 | 1.84 | octanal |
| 17 | 1.04 | Unknown |
| 18 | 1.01 | undecane |
| 19 | 1.08 | nonanal |
| 20 | 2.77 | 2,2,4,4-tetramethyl pentanoic acid |
| 21 | 1.39 | dodecane |
| 22 | 1.20 | siloxane (artifact) |
| 23 | 4.25 | N,N-dibutyl formamide |
| 24 | 0.71 | siloxane (artifact) |
| 25 | 0.59 | siloxane (artifact) |
| 26 | 0.33 | siloxane (artifact) |
| 27 | 0.62 | siloxane (artifact) |

As indicated, the sample exhibited prominent peaks for acetic acid and formic acid, but lacked significant amounts of the more odorous aldehyde and ketone compounds.

### EXAMPLE 3

The substrate was formed from a blend of pulp fibers containing 55.6 wt. % LL-19 and 44.4 wt. % Aracruz Eucalyptus. Kymene® 557LX (Hercules, Inc.) was also added to the pulp furnish. LL-19 is a bleached Northern softwood pulp and Aracruz Eucalyptus is a bleached hardwood pulp. 8 ounces of soda ash were added to the pulp furnish in the pulper per 2332 pounds of pulp so that the pH of the pulp furnish was approximately 7.0. A web was formed and then saturated off-line with an aqueous solution of soda ash (0.1 wt %) having a pH of 10.5. The pH of the paper was measured and determined to be 7.1.

### EXAMPLE 4

The substrate was formed from a blend of pulp fibers containing 55.6 wt. % LL-19 and 44.4 wt. % Aracruz Eucalyptus. Kymene® 557LX (Hercules, Inc.) was also added to the pulp furnish. LL-19 is a bleached Northern softwood pulp and Aracruz Eucalyptus is a bleached hardwood pulp. 8 ounces of soda ash were added to the pulp furnish in the pulper per 2332 pounds of pulp so that the pH of the pulp furnish was approximately 7.0. A web was formed and then saturated off-line with an aqueous solution of soda ash (0.1 wt %) having a pH of 10.5. Thereafter, the web was saturated with a dispersion that contained 37.3 wt. % Hycar® 26469 (Noveon, Inc.), 45.5 wt. % of Michelman® 4983R, 16.6% pigment, and a small quantity of Nopcote DC-100A. The pH of the paper was measured and determined to be 7.2.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A medical packaging substrate comprising a fibrous web, wherein the web is formed from a cellulosic fibrous material treated with a pH modifier so that the pH of the material is about 7.0 or more, further wherein the fibrous web is impregnated with a binder composition, the impregnated fibrous web having a Gurley porosity of from about 10 to about 120 seconds per 100 cubic centimeters.

2. The medical packaging substrate of claim 1, wherein the cellulosic fibrous material comprises Kraft pulp fibers.

3. The medical packaging substrate of claim 1, wherein the pH of the cellulosic fibrous material is from about 7.0 to about 9.0.

4. The medical packaging substrate of claim 1, wherein the pH of the cellulosic fibrous material is from about 7.5 to about 8.0.

5. The medical packaging substrate of claim 1, wherein the pH modifier is sodium carbonate, sodium bicarbonate, or a mixture thereof.

6. The medical packaging substrate of claim 1, wherein the cellulosic fibrous material is further treated with a wet-strength agent.

7. The medical packaging substrate of claim 6, wherein the wet-strength agent is a polyamine-epichlorohydrin, polyamide-epichlorohydrin, polyamide-amine epichlorohydrin, or a mixture thereof.

8. The medical packaging substrate of claim 1, wherein the binder composition includes a carboxylated polyacrylate.

9. The medical packaging substrate of claim 8, wherein the binder composition further includes a lower alkene polymer.

10. The medical packaging substrate of claim 1, wherein the medical packaging substrate has a Gurley porosity of from about 20 to about 80 seconds per 100 cubic centimeters.

11. The medical packaging substrate of claim 1, wherein the medical packaging substrate has a Gurley porosity of from about 30 to about 60 seconds per 100 cubic centimeters.

12. The medical packaging substrate of claim 1, wherein the substrate has a pH of greater than about 7.0.

13. A method for forming a medical packaging substrate, the method comprising:
forming a suspension into a fibrous web, the suspension comprising a cellulosic fibrous material; and
impregnating the fibrous web with a binder composition;
wherein the suspension, the fibrous web prior to impregnation with the binder composition, or both are treated with a pH modifier so that the pH of the cellulosic fibrous material is about 7.0 or more.

14. The method of claim 13, wherein the cellulosic fibrous material comprises Kraft pulp fibers.

15. The method of claim 13, wherein the pH of the cellulosic fibrous material is from about 7.0 to about 9.0.

16. The method of claim 13, wherein the pH of the cellulosic fibrous material is from about 7.5 to about 8.0.

17. The method of claim 13, wherein the pH modifier is sodium carbonate, sodium bicarbonate, or a mixture thereof.

18. The method of claim 13, wherein the cellulosic fibrous material is treated with a wet-strength agent.

19. The method of claim 18, wherein the wet-strength agent is a polyamine-epichlorohydrin, polyamide-epichlorohydrin, polyamide-amine epichlorohydrin, or a mixture thereof.

20. The method of claim 13, wherein the binder composition includes a carboxylated polyacrylate and a lower alkene polymer.

21. The method of claim 13, wherein the suspension is treated with the pH modifier.

22. The method of claim 13, wherein the fibrous web is applied with a solution containing the pH modifier.

23. A method for sterilizing an item, the method comprising:
enclosing the item within a medical package, wherein the medical package is formed from a substrate that comprises a cellulosic fibrous material that is impregnated with a binder composition, the substrate exhibiting an initial machine direction and cross machine direction tensile strength; and
treating the substrate with ozone, wherein the ozone-treated substrate exhibits a machine direction tensile strength that is no more than about 40% less than the initial machine direction tensile strength.

24. The method of claim 23, wherein the ozone-treated substrate exhibits a machine direction tensile strength that is no more than about 30% less than the initial machine direction tensile strength.

25. The method of claim 23, wherein the ozone-treated substrate exhibits a cross machine direction tensile strength that is no more than about 40% less than the initial cross machine direction tensile strength.

26. The method of claim 23, wherein the ozone-treated substrate exhibits a cross machine direction tensile strength that is no more than about 30% less than the initial cross machine direction tensile strength.

27. The method of claim 23, wherein the ozone-treated substrate exhibits a machine direction tensile strength of at least about 4 kilograms per 15 millimeters and a cross machine direction tensile strength of at least about 2 kilograms per 15 millimeters.

28. The method of claim 23, wherein the ozone-treated substrate exhibits a machine direction tensile strength of at least about 5 kilograms per 15 millimeters and a cross machine direction tensile strength of at least about 3 kilograms per 15 millimeters.

29. The method of claim 23, wherein the ozone-treated substrate exhibits a machine direction elongation of at least about 1% and a cross machine direction elongation of at least about 4%.

30. The method of claim 23, wherein the ozone-treated substrate exhibits a machine direction elongation of at least about 2% and a cross machine direction elongation of at least about 5%.

31. The method of claim 23, wherein the cellulosic fibrous material comprises Kraft pulp fibers.

32. The method of claim 23, wherein the pH of the cellulosic fibrous material is from about 7.0 to about 9.0.

33. The method of claim 23, wherein the cellulosic fibrous material is treated with a wet-strength agent selected from the group consisting of polyamine-epichlorohydrin, polyamide-epichlorohydrin, polyamide-amine epichlorohydrin, or a mixture thereof.

34. The method of claim 23, wherein the binder composition includes a carboxylated polyacrylate and a lower alkene polymer.

35. The method of claim 23, wherein the ozone has a temperature of from about 20° C. to about 35° C.

36. The method of claim 23, wherein the ozone is humidified.

37. The method of claim 23, wherein the package is formed by sealing the substrate to a base component.

38. The method of claim 37, wherein the package exhibits a peel strength of at least about 0.70 pounds per inch after ozone treatment.

39. The method of claim 37, wherein the package exhibits a peel strength of from about 1.2 to about 2.0 pounds per inch after ozone treatment.
